SEQUENCE LISTING (11 sequences)

SEQ ID NO: 1 - protein, 494 aa, RIGIDOPORUS MICROPORUS
SVGPVADIPIVNANLSPDGFTRTTVLAGGTFPGPLIVGNKGDNFKLNVVDQLTDANQLKTTTIHWHGFFQHGTNWADGPAFVNQCPIASGNSFLYDFSAADQAGTFWYHSHLSTQYCDGLRGAFVVYDPSDPNASLYDVDNESTVITLADWYHTLARLGARFPTPDSTLINGLGRFAGGPASDLSVITVESGKRYRFRLVSISCDPNYTFSIDGHDMTIIEVNGINHDALSVDSIQIFAGQRYSFVLNANQAVGNYWIRANPNIGTRGFSGGINSAILRYVGADAVEPTTSQGTSTKPLVETNLHPSQNPGAVGSPTPGGVDLALNLALGFAGGSFTINGATFTSPTVPVLLQILSGAQSATDLLPSGSVFTLPGDSTIEISMPAGVAGGPHPFHLHGHAFDVVRASGSSTYNYANPVRRDVVSLGAAGDNVTIRFKTDNPGPWFLHCHIDWHLEAGLAIVFAEDTPNTAAINPVPQAWSDLCPIYNALAESDH

SEQ ID NO: 2 - protein, 515 aa, RIGIDOPORUS MICROPORUS
MPSFSTLSAFVTVALALGAFASVGPVADIPIVNANLSPDGFTRTTVLAGGTFPGPLIVGNKGDNFKLNVVDQLTDANQLKTTTIHWHGFFQHGTNWADGPAFVNQCPIASGNSFLYDFSAADQAGTFWYHSHLSTQYCDGLRGAFVVYDPSDPNASLYDVDNESTVITLADWYHTLARLGARFPTPDSTLINGLGRFAGGPASDLSVITVESGKRYRFRLVSISCDPNYTFSIDGHDMTIIEVNGINHDALSVDSIQIFAGQRYSFVLNANQAVGNYWIRANPNIGTRGFSGGINSAILRYVGADAVEPTTSQGTSTKPLVETNLHPSQNPGAVGSPTPGGVDLALNLALGFAGGSFTINGATFTSPTVPVLLQILSGAQSATDLLPSGSVFTLPGDSTIEISMPAGVAGGPHPFHLHGHAFDVVRASGSSTYNYANPVRRDVVSLGAAGDNVTIRFKTDNPGPWFLHCHIDWHLEAGLAIVFAEDTPNTAAINPVPQAWSDLCPIYNALAESDH

SEQ ID NO: 3 - DNA, 1485 bp, RIGIDOPORUS MICROPORUS
tccgtcgggcccgtggctgacattcccattgtcaacgctaacctctctccagatggtttcactcgtaccactgttctcgcaggtggaaccttccctggacccctcatcgtcggaaataagggcgataacttcaaacttaatgtcgtagaccaactcaccgatgccaatcaactgaagaccacaaccattcactggcacggtttcttccaacacggcaccaactgggcggatgggcccgcattcgtaaaccagtgcccgatcgcttctggtaactccttcttgtacgatttctccgctgccgaccaagctggcacattctggtaccacagtcatctttcgacgcagtactgcgatggtttgcgtggggccttcgtggtgtacgatcccagtgaccccaatgcgagcttgtatgacgtcgataatgagagcactgttattacccttgcggattggtatcacaccttggcacggttgggtgctaggttcccgactcctgactcaactttgatcaatggcctcgggcggtttgctggaggacctgcttcggacttgtccgtcattactgtggaatcgggtaaacgatatcgtttccgtcttgtatccatctcttgcgatcccaattatacattctccattgatggtcacgacatgacaatcattgaagtcaatggtattaaccacgacgcattgtctgttgattcgatccaaatattcgccggtcaacggtactccttcgtgctcaatgcaaaccaagccgtgggcaactactggatccgcgccaaccccaacatcggtaccagagggttctcgggcggcattaactcggccattctccggtatgtcggtgccgacgcagtcgaacccacaacttctcaaggtaccagcaccaaacctctcgtcgaaaccaacttgcatcccagccaaaacccgggtgctgtcgggtctcccactccaggtggtgtcgaccttgctttgaacttggcccttggattcgccggaggatcattcaccatcaacggcgctaccttcacttctcccaccgttcctgtccttctccaaattctcagtggtgcacaatcagcgacagatttgcttccgtcaggcagtgtcttcactcttccaggagattctaccatcgagatcagcatgcctgctggtgtcgctggtggtccccatcccttccacttgcatggtcacgctttcgacgtcgttcgcgcctccggtagctcaacttacaactacgctaatcctgttcgccgtgatgttgtctcccttggtgccgctggtgacaatgttacgatcagattcaagaccgacaacccgggaccttggttcctccattgtcacattgactggcatctcgaagccggattggccattgtctttgctgaagacacgcccaacactgccgcgataaacccagttccacaggcttggagtgacctgtgccccatctataatgctcttgctgagtctgatcattaa

SEQ ID NO: 4 - DNA, 1548 bp, RIGIDOPORUS MICROPORUS
atgccttctttctcaaccctctctgcctttgtgactgtcgccctcgctcttggggcatttgcctccgtcgggcccgtggctgacattcccattgtcaacgctaacctctctccagatggtttcactcgtaccactgttctcgcaggtggaaccttccctggacccctcatcgtcggaaataagggcgataacttcaaacttaatgtcgtagaccaactcaccgatgccaatcaactgaagaccacaaccattcactggcacggtttcttccaacacggcaccaactgggcggatgggcccgcattcgtaaaccagtgcccgatcgcttctggtaactccttcttgtacgatttctccgctgccgaccaagctggcacattctggtaccacagtcatctttcgacgcagtactgcgatggtttgcgtggggccttcgtggtgtacgatcccagtgaccccaatgcgagcttgtatgacgtcgataatgagagcactgttattacccttgcggattggtatcacaccttggcacggttgggtgctaggttcccgactcctgactcaactttgatcaatggcctcgggcggtttgctggaggacctgcttcggacttgtccgtcattactgtggaatcgggtaaacgatatcgtttccgtcttgtatccatctcttgcgatcccaattatacattctccattgatggtcacgacatgacaatcattgaagtcaatggtattaaccacgacgcattgtctgttgattcgatccaaatattcgccggtcaacggtactccttcgtgctcaatgcaaaccaagccgtgggcaactactggatccgcgccaaccccaacatcggtaccagagggttctcgggcggcattaactcggccattctccggtatgtcggtgccgacgcagtcgaacccacaacttctcaaggtaccagcaccaaacctctcgtcgaaaccaacttgcatcccagccaaaacccgggtgctgtcgggtctcccactccaggtggtgtcgaccttgctttgaacttggcccttggattcgccggaggatcattcaccatcaacggcgctaccttcacttctcccaccgttcctgtccttctccaaattctcagtggtgcacaatcagcgacagatttgcttccgtcaggcagtgtcttcactcttccaggagattctaccatcgagatcagcatgcctgctggtgtcgctggtggtccccatcccttccacttgcatggtcacgctttcgacgtcgttcgcgcctccggtagctcaacttacaactacgctaatcctgttcgccgtgatgttgtctcccttggtgccgctggtgacaatgttacgatcagattcaagaccgacaacccgggaccttggttcctccattgtcacattgactggcatctcgaagccggattggccattgtctttgctgaagacacgcccaacactgccgcgataaacccagttccacaggcttggagtgacctgtgccccatctataatgctcttgctgagtctgatcattaa

SEQ ID NO: 5 - DNA, 2606 bp, RIGIDOPORUS MICROPORUS
gatcctagcatggttccttcctctcccaaacatgtcgttcccaattcataccaagttgtacttgcacaactggcattgatggcgcacgtataagagggatggggtgtgaatccgtctccctcatcccgcttcttcaactcgggctactccattgcattcgaccaccagttgagacatgccttctttctcaaccctctctgcctttgtgactgtcgccctcgctcttggggcatttgcctccgtcgggcccgtggctgacattcccattgtcaacgctaacctctctccagatggtttcactcgtaccactgttctcgcaggtggaaccttccctggacccctcatcgtcggaaataaggtcggtccatatgaccgctactttcctcaggagaaattttgacttcttgcgcgcacagggcgataacttcaaacttaatgtcgtagaccaactcaccgatgccaatcaactgaagaccacaaccattgtaggttttgcattgttccctcagcttcgtgtctcatttcctctcgttagcactggcacggtttcttccaacacggcaccaactgggcggatgggcccgcattcgtaaaccagtgcccgatcgcttctggtaactccttcttgtacgatttctccgctgccgaccaagctggtaagtctggcacagtgccagagccgaggtaggcaagccgagctgaccatcttcacacaggcacattctggtaccacagtcatctttcgacgcagtactgcgatggtttgcgtggggccttcgtggtgtacgatcccagtgaccccaatgcgagcttgtatgacgtcgataatggtacgaacttttcttcataccccttcccgaacaccgttgaccgtccatacttcttttcagagagcactgttattacccttgcggattggtatcacaccttggcacggttgggtgctaggttcccgtgagtcacatgttcgcgttcccctgtggtttatgtcattcatcattcttttcccaggactcctgactcaactttgatcaatggcctcgggcggtttgctggaggacctgcttcggacttgtccgtcattactgtggaatcgggtaaacggtatgtttatgatgtgtaccttgaacacaaaataagcattgattcaacccatcctttcttacctcatttagatatcgtttccgtcttgtatccatctcttgcgatcccaattatacattctccattgatggtcacgacatgacaatcattgaagtcaatggtattaaccacgacgcattgtctgttgattcgatccaaatattcgccggtcaacggtactccttcgtggtatgtcccccacgctcccttcataactctcttattcacatgatcattctcagctcaatgcaaaccaagccgtgggcaactactggatccgcgccaaccccaacatcggtaccagagggttctcgggcggcattaactcggccattctccgatatgtcggtgccgacgcagtcgaacccacaacttctcaaggtaccagcaccaaacctctcgtcgaaaccaacttgcatcccagccaaaacccgggtgctgtaagtcccaagcgttatctccttgttttcggaagtcctcatctattgttttgtaggtcgggtctcccactccaggtggtgtcgaccttgctttgaacttggcccttggattcgtacgtacacattttatccagttcctgaatgtgttcctcatcttccgtttaggccggaggatcattcaccatcaacggcgctaccttcacttctcccaccgttcctgtccttctccaaattctcagtggtgcacaatcagcgacagatttgcttccgtcaggcagtgtcttcactcttccaggagattctaccatcgagatcagcatgcctgctggtgtcgctggtggtccccatcccttccacttgcatggtgtaggtccctcaattattcatacttcctaatgctcacgaatccttctccagcacgctttcgacgtcgttcgcgcctccggtagctcaacttacaactacgctaatcctgttcgccgtgatgttgtctcccttggtgccgctggtgacaatgttacgatcagattcaaggtaagctgatagatggatcctcgggtggcttcgcttgctggcgaagtgagcagaccgacaacccgggaccttggttcctccattgtcacattgactggcatctcgaagccggattggccattgtctttgctgaagacacgcccaacactgccgcgataaaccctgttccacgtacgttccgttttaccgagcgcacgttcttctctcattgtttacttctcccacagaggcttggagtgacctgtgccccatctataatgctcttgctgagtctgatcattaaatcagaagaacaagggttacagacgagacaaggactaaaatgaatacctactctctccttgcgattctatctattcttctatttactctttatctttttggttttgaccaactgtggaaattggtcatgcaatttttcttgtctcgaaatcggaacaatgtgtaagtagctacttgaaatgaaaatcctgtccagaatgttgcact

SEQ ID NO: 6 - DNA, 38 bp, Artificial Sequence
  Synthetic primer
ggttcttgccacagtcacgacttttttttttttttttt

SEQ ID NO: 7 - DNA, 21 bp, Artificial Sequence
  Synthetic primer
ggttcttgccacagtcacgac

SEQ ID NO: 8 - DNA, 20 bp, Artificial Sequence
  Synthetic primer
ggcccngtngcngayathcc

SEQ ID NO: 9 - DNA, 29 bp, Artificial Sequence
  Synthetic primer
actcgtaccactgttctcgcaggtggaac

SEQ ID NO: 10 - DNA, 28 bp, Artificial Sequence
  Synthetic primer
gaaaccatctggagagaggttagcgttg

SEQ ID NO: 11 - protein, 9 aa, Artificial Sequence
  N-terminal fragment of Lcc3
SVGPVADIP

(19) United States
(12) Patent Application Publication
(10) Pub. No.: US 2010/0184186 A1
(43) Pub. Date: Jul. 22, 2010

(54) NOVEL RIGIDOPORUS MICROPORUS LACCASE

(75) Inventors: Po-Ting Chen, Taipei (TW); Chii-Gong Tong, Sinying City (TW); Tuan-Hua David Ho, Taipei (TW); Su-May Yu, Taipei (TW)

Correspondence Address: OCCHIUTI ROHLICEK & TSAO, LLP, 10 FAWCETT STREET, CAMBRIDGE, MA, 02138, US

(73) Assignee: Academia Sinica, Taipei (TW)

(21) Appl. No.: 12/691,904

(22) Filed: Jan. 22, 2010

(30) Foreign Application Priority Data
Jan. 22, 2009 (TW) 98102621

(51) Int. Cl. C12N 9/02; C07H 21/04; C12N 15/63; C12N 1/15

(52) U.S. Cl. 435/189; 536/23.2; 435/320.1; 435/254.11

(57) ABSTRACT

An isolated R. microporus laccase, a nucleic acid encoding the laccase, and a method of preparing it in a cell.

### RELATED APPLICATION

This application claims priority to Taiwanese Patent Application No. 98102621, filed on Jan. 22, 2009, the content of which is hereby incorporated by reference in its entirety.

### BACKGROUND OF THE INVENTION

Laccases (also known as bensenebiol:oxygen oxidoreductase; EC 1.10.3.2) are multi-copper-containing oxidases found in various organisms, e.g., insect, plant, and fungi. They catalyze oxidation of a broad range of compounds, e.g., diphenol, polyphenol, diamine, and aromatic amine. Many of these compounds are important raw materials for making various industrial products. Others are toxic components contained in industrial wastes. As such, laccases have great potentials in industrial applications, including biopulping, biobleaching, food processing, bioremediation, and wastewater treatment.

### SUMMARY OF THE INVENTION

The present invention is based on an unexpected discovery of a novel laccase (i.e., Lcc35) from R. microporus BCRC 35318 that exhibits high laccase activity.

Accordingly, one aspect of this invention provides an isolated polypeptide containing an amino acid sequence at least 85% (e.g., 90% or 95%) identical to SEQ ID NO:1, which refers to the amino acid sequence of the mature form of Lcc35. The polypeptide of this invention can be mature Lcc35 (SEQ ID NO:1) or precursor Lcc35 (SEQ ID NO:2).

Another aspect of the invention provides an isolated nucleic acid (e.g., an expression vector) including a nucleotide sequence that encodes any of the polypeptides mentioned above. The nucleotide sequence can be SEQ ID NO:3, coding for SEQ ID NO:1, or SEQ ID NO:4, coding for SEQ ID NO:2. It can be linked operatively with a suitable promoter for expressing the encoded polypeptide in a host cell.

The terms “isolated polypeptide” and “isolated nucleic acid” used herein respectively refer to a polypeptide and a nucleic acid substantially free from naturally associated molecules. A preparation containing the polypeptide or nucleic acid is deemed as “an isolated polypeptide” or “an isolated nucleic acid” when the naturally associated molecules in the preparation constitute at most 20% by dry weight. Purity can be measured by any appropriate method, e.g., column chromatography, polyacrylamide gel electrophoresis, and HPLC.

Also within the scope of this invention is a method of preparing any of the polypeptides disclosed herein. This method includes at least three steps: (i) providing a host cell containing a nucleic acid for expressing the polypeptide, (ii) culturing the cell in a medium under conditions allowing expression of the polypeptide, and (iii) collecting the cells, the medium, or both for isolation of the polypeptide. The laccase activity of the polypeptide thus isolated can be confirmed by routine methods, e.g., those described in Example 1 below. When R. microporus is used as the host, the medium can include an inducer (e.g., 4-hydroxybenzoic acid, rice straw, veratryl alcohol, or ferulic acid) to enhance production of the polypeptide.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The drawings are first described.

FIG. 1 is a diagram showing the genomic sequence of the Lcc35 gene (SEQ ID NO:5) and the amino acid sequence of precursor Lcc35 (SEQ ID NO:2). Uppercase regions refer to the 5′ and 3′ untranslated regions, uppercase and boldface regions refer to coding regions, and lowercase regions refer to intronic sequences. The highlighted region refers to the signal peptide and the bracketed region is the N-terminal portion of the mature Lcc35. Five potential glycosylation sites, i.e., N-X-S/T (X being any amino acid residue), are underlined. Amino acid residues that are in type-1, type-2, and type-3 copper domain centers are marked by underneath numbers 1, 2, and 3, respectively. Other functionally essential amino acid residues are boldfaced and italicized.

FIG. 2 is a diagram showing a phylogenetic tree including Lcc35 and other fungal laccases. The phylogenetic tree was prepared using the CLUSTAL program (MEGA 4).

FIG. 3 is a diagram showing the effect of pH on Lcc35 laccase activity. (A) Relative laccase activities at various pH conditions using ABTS (); SGZ (▴), or lignin (▾) as the substrate. (B) De-colorization of RBBR by Lcc35 at various pH conditions. (C) Lcc35 stability at various pH conditions.

FIG. 4 is a diagram showing the effect of temperature on Lcc35 laccase activity using ABST as the substrate. (A) Lcc35 activities at various temperatures. (B) Lcc35 thermostability at 30° C. (); 40° C. (∘); 50° C. (▾); 60° C. (Δ); or 70° C. (▪).

### DETAILED DESCRIPTION OF THE INVENTION

Described herein is laccase Lcc35 isolated from R. microporus. The amino acid sequence of this enzyme in precursor form (SEQ ID NO:2) and its coding sequence (SEQ ID NO:4) are shown below (see also GenBank Accession Number FJ002275):

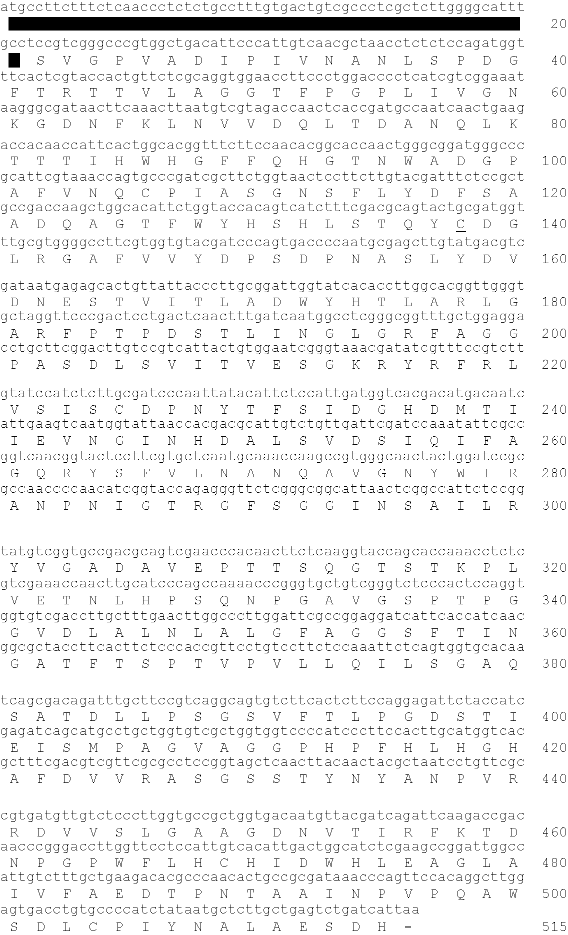

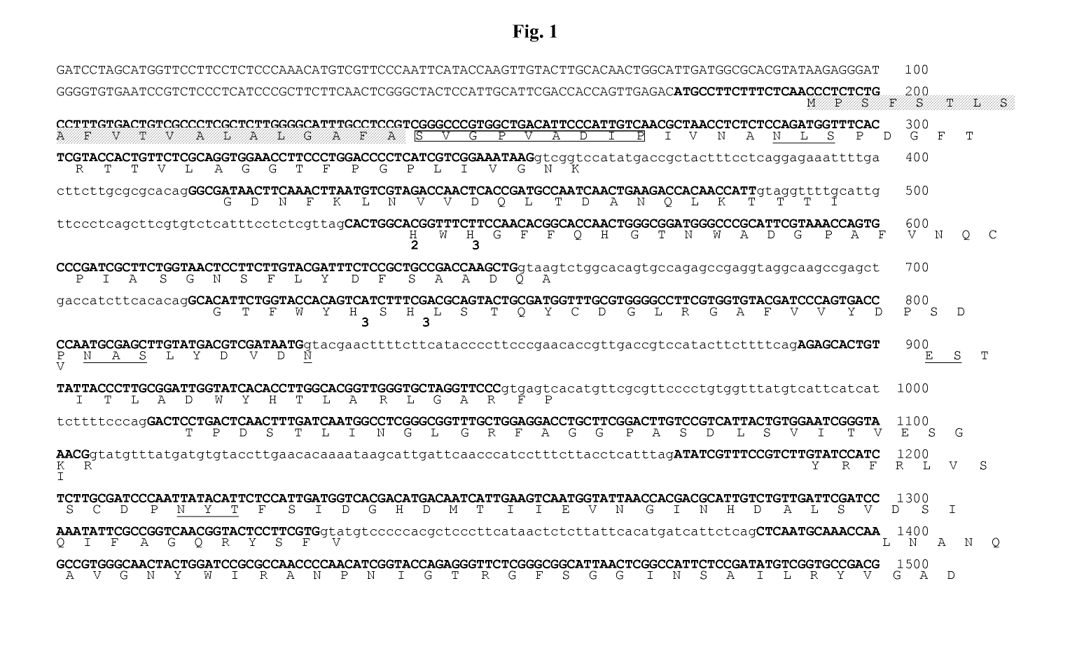

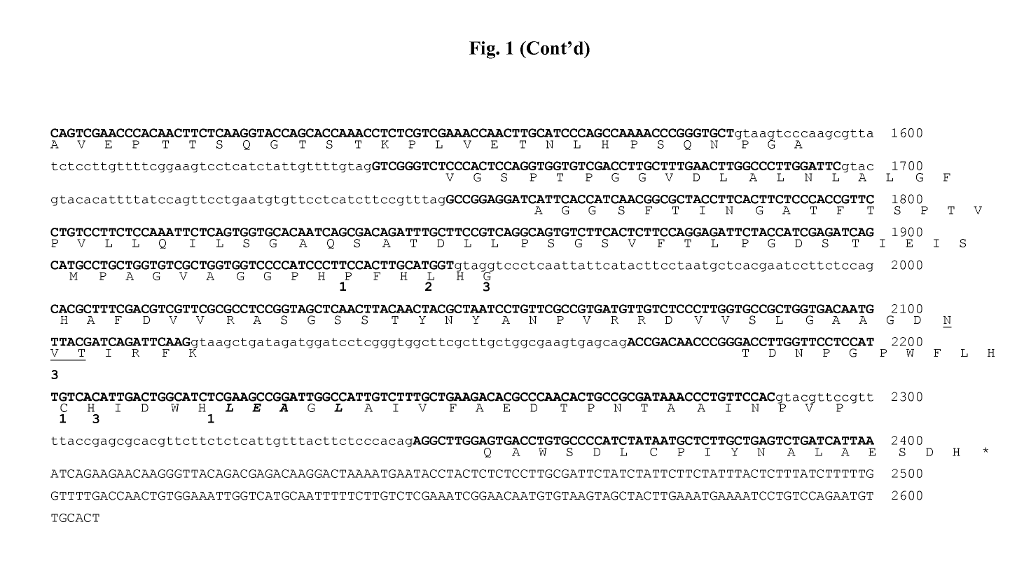

Precursor Lcc35 contains a signal peptide located at the N-terminal region of 1-21 (highlighted). See also FIG. 1. Five potential glycosylation sites (Asn-X-Ser/Ter) were identified in this enzyme according to the method described in Gavel et al., Protein Eng. 1990, 3(5):433-442. See positions 35, 154, 162, 228, and 452 (all in boldface) in the above amino acid sequence.

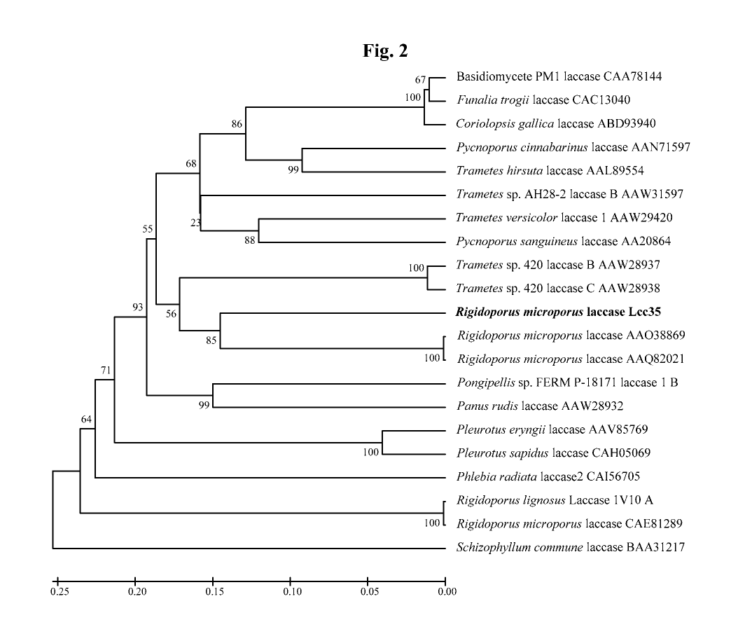

A phylogenetic tree was obtained by comparing the structure of Lcc35 with other fungal laccases with the CLUSTAL program (MEGA 4). See FIG. 2. Based on their sequence alignments, it has been determined that two disulfide bonds can be formed in Lcc35, one between Cys106 and Cys504, and the other between Cys138 and Cys225 (see the underlined Cys residues in the above amino acid sequence). In Lcc35, Cys469 is deemed to be a ligand in a mononuclear type-1 copper domain center. Further, domains TTIHWHGFF and PHPFHLHGH in Lcc35 are deemed essential to its enzymatic activity. Other functionally important domains or residues are shown in FIG. 1 or can be determined based on the laccase structure-function correlation described in Stoj, C. S., and Kosman, D. J. (2005) Copper Oxidases, in Encyclopedia of Bioinorganic Chemistry, 2nd Ed., R. B. King, ed, John Wiley.

Also described herein are functional variants of Lcc35 that share at least 85% (e.g., 90%, 95%, or 98%) sequence identity to SEQ ID NO:1. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

The functional variants of Lcc35 can contain conservative mutations inside the functional domains or at the essential residue positions as described above. A mutation is conservative when the amino acids used for the substitutions have structural or chemical characteristics similar to those of the corresponding replaced amino acids. Examples of conservative substitutions can include: substitution of Ala with Gly or Val, substitution of Arg with His or Lys, substitution of Asn with Glu, Gln, or Asp, substitution of Asp with Asn, Glu, or Gln, substitution of Cys with Ser or Ala, substitution of Gln with Asn, Glu, or Asp, substitution of Glu with Gly, Asn, Gln, or Asp, substitution of Gly with Val or Ala, substitution of substitution of Ile with Leu, Met, Val, or Phe, substitution of Leu with Ile, Met, Val, or Phe, substitution of Lys with His or Arg, substitution of Met with Ile, Leu, Val, or Phe, substitution of Phe with Trp, Tyr, Met, Ile, or Leu, substitution of Ser with Thr or Ala, substitution of Thr with Ser or Ala, substitution of Trp with Phe or Tyr, substitution of Tyr with His, Phe, or Trp, and substitution of Val with Met, Ile, Leu, or Gly.

Conservative mutations in the functional domains would not abolish the enzymatic activity of the resultant Lcc35 variants. On the other hand, domains not essential to the laccase activity are tolerable to mutations as amino acid substitutions within these domains are unlikely to greatly affect enzyme activity.

Lcc35 and any of its functional variants can be prepared by conventional recombinant technology. Generally, a coding sequence for Lcc35 can be isolated from R. microporus via routine molecular cloning technology. Nucleotide sequences coding for the Lcc35 variants can be prepared by modifying the Lcc35-coding sequence. Any of the coding sequences can then be inserted into an expression vector, which contains a suitable promoter in operative linkage with the coding sequence.

As used herein, a “vector” refers to a nucleic acid molecule capable of transporting another nucleic acid to which it has been linked. The vector can be capable of autonomous replication or integrate into a host DNA. Examples of the vector include a plasmid, cosmid, or viral vector. An expression is a vector in a form suitable for expression of a target nucleic acid in a host cell. Preferably, an expression vector includes one or more regulatory sequences operatively linked to a target nucleic acid sequence to be expressed. The term “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of transcription of RNA desired, and the like.

The term “promoter” refers to a nucleotide sequence containing elements that initiate the transcription of an operably linked nucleic acid sequence in a desired host cell. At a minimum, a promoter contains an RNA polymerase binding site. It can further contain one or more enhancer elements which, by definition, enhance transcription, or one or more regulatory elements that control the on/off status of the promoter. When E. coli is used as the host, representative E. coli promoters include, but are not limited to, the β-lactamase and lactose promoter systems (see Chang et al., Nature 275:615-624, 1978), the SP6, T3, T5, and T7 RNA polymerase promoters (Studier et al., Meth. Enzymol. 185:60-89, 1990), the lambda promoter (Elvin et al., Gene 87:123-126, 1990), the trp promoter (Nichols and Yanofsky, Meth. in Enzymology 101:155-164, 1983), and the Tac and Trc promoters (Russell et al., Gene 20:231-243, 1982). When yeast is used as the host, exemplary yeast promoters include 3-phosphoglycerate kinase promoter, glyceraldehyde-3-phosphate dehydrogenase (GAPDH) promoter, galactokinase (GAL1) promoter, galactoepimerase promoter, and alcohol dehydrogenase (ADH) promoter. Promoters suitable for driving gene expression in other types of microorganisms are also well known in the art. Examples of mammalian cell promoters include, but are not limited to, CMV promoter, SV40 promoter, and actin promoter.

The expression vector described above is then introduced into a suitable host (e.g., E. coli, yeast, an insect cell, and a mammalian cell) for expressing of Lcc35 or its variant. Positive transformants/transfectants are selected and over-expression of the enzyme can be confirmed by methods known in the art, e.g., immune-blotting or enzymatic activity analysis. A host cell carrying the expression vector is then cultured in a suitable medium under suitable conditions for laccase production. The culture medium or the cells are harvested for isolation of the enzyme. When the enzyme is expressed in precursor form, i.e., containing an N-terminal signal peptide, it is preferred that the culture medium be collected for enzyme isolation. The activity of the isolated enzyme can then be confirmed by a conventional assay, e.g., those described in Example 1 below.

Alternatively, Lcc35 or a variant thereof can be prepared by culturing a suitable R. microporus strain (e.g., BCRC 35318 provided by Bioresource Collection and Research Center, Hshinchu, Taiwan) via a traditional method. See, e.g., Example 2 below. The enzyme can be purified from the culture medium.

Lcc35 and its functional variants can oxidize both phenolic and non-phenolic lignin related compounds, as well as highly recalcitrant environmental pollutants. As such, they have broad biotechnological and industrial applications. For example, they can be used to detoxify industrial effluents, particularly those from the paper and pulp, textile and petrochemical industries. In addition, Lcc35 and its variants can be used to detect and clean up herbicides, pesticides, and certain explosives in environmental water or soil. They also can be used in treating industrial wastewater. Further, given their capacity of removing xenobiotic substances and producing polymeric products, Lcc35 and the variants can serve as bioremediation agents to reduce environmental contamination. The laccases can also be used in food industry to remove phenolic compounds in food products, thereby enhancing food quality.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### Example 1

### Identification, Cloning, and Characterization of Lcc35

Identification of Laccase Lcc35 from R. microporus BCRC 35318

A potato dextrose agar (PDA) plate containing Remazol brilliant blue R (RBBR) was used to determining the activity of laccase secreted by R. microporus BCRC 35318, following the method described in, e.g., Kiiskinen et al., J. Appl. Microbiol. 2004, 97, (3), 640-646. Briefly, cells of R. microporus BCRC 35318 were placed on top of the PDA plate and incubated at 30° C. for 5 to 8 days to allow formation of R. microporus colonies. Large halos surrounding the colonies were observed, indicating that R. microporus BCRC 35318 secrets a laccase with high enzymatic activity.

To isolate the laccase from R. microporus BCRC 35318, eight-day-old mycelial disks (8 mm in diameter) collected from the PDA plate mentioned above were inoculated into a basal medium containing (per liter) 10 g glucose, 0.22 g ammonium tartrate, 0.9 g K₂HPO₄, 0.1 g KH₂PO₄, 0.05 g MgSO₄.7H₂O, 0.5 g CaCl₂, 0.01 g Thiamine HCl, and 10 ml solution (0.08 g CuSO₄.5H₂O, 0.07 g MnSO₄.4H₂O, 4.3 g ZnSO₄.7H₂O and 0.05 g FeSO₄.7H₂O per liter). The pH of the medium was adjusted to 5.5. After being cultured for 10 days at 30° C. in a rotary shaker with a speed setting of 125 rpm, the supernatant of the fungal culture thus obtained was collected and passed through a 0.45 mm filter (Nalgene) to remove fungal cells. The filtrate was mixed with 100% ammonium sulfate for protein precipitation. The precipitated proteins were dissolved in 40 ml of a 10 mM sodium acetate buffer (pH 6.0) and the resultant solution was dialyzed against the same acetate buffer overnight to remove co-precipitated ammonium sulfate and then concentrated to 4 ml by ultracentrifugal filteration with a molecular cutoff of 10 kDa (Amicon).

The total proteins in the concentrated solution were analyzed by SDS-PAGE, according to the method described in Laemmli et al., Nature 1970, 227 (5259):680-685. Briefly, the solution was mixed with a sample buffer containing 1% SDS and 2.56% 2-mercaptorthanol (2-ME) at a volume ratio of 1:1. The mixture was boiled at 100° C. for 3 min and then subjected to SDS-PAGE. Upon Coomassie blue staining, a major protein band was observed at a position corresponding to molecule weight 55 kDa. It was estimated that this protein constituted about 90% of the total proteins in the supernatant.

The laccase activity of this 55kDa protein was determined by an [2,2-azinobis(3-ethylbenzathiazoline-6-sulfonic acid)] (ABTS) overlay activity assay. See Lebendiker M BASIC-NATIVE GEL Protocol, wolfson.huji.ac.il/purification/Protocols/PAGE_Basic.html. A basic-native PAGE gel containing the 55 kDa protein was incubated in an ABTS reaction buffer (1 mM, pH 3) at room temperature. As indicated by a color change around the 55 kDa protein band, this protein (designated Lcc35) was found to exhibit high laccase activity.

### Characterization of Lcc35

### (i) Isoelectric Focusing Point

First, the isoelectric focusing point of Lcc35 was determined with PhastGel IEF 3-9 (GE Healthcare), following the method described in Hackler et al., Anal. Biochem. 1995, 230, (2), 281-289. Briefly, the Lcc35-containing solution mentioned above was loaded on a PhastGel IEF 3-9 gel. After electrophoresis, the gel was stained with PhastGel Blue R. Lcc35 was found to have a pI value of 3.98.

### (ii) Optimal pH and pH Stability

Next, the laccase activities of Lcc35 under various pH conditions were determined, following the method described in Lu et al., Appl. Microbiol. Biotechnol., 2007, 74, (6), 1232-1239. 0.01 ml of the Lcc35-containing solution was mixed with (i) 0.49 ml of a 50 mM reaction buffer of glycine-HCl (pH 2.0 to 3.0), sodium acetate (pH 4.0 to 6.0), or sodium phosphate (pH 7.0 to 8.0) and (ii) 0.5 ml of a substrate solution containing 2 mM ABTS, 1 mM [N,N′-bis(3,5-dimethoxy-4-hydroxybenzylidene)hydrazine] (SGZ), or 0.04% RBBR. After being incubated at 30° C. for 1 min (when ABST or SGZ was the substrate) or for 4 hours (when RBBR was the substrate), each of the reaction mixtures was examined with a spectrophotometer to determine its optical density at 420 nm (when ABST was the substrate), at 530 nm (when SGZ was the substrate), or at 585 nm (when RBBR was the substrate). One unit of laccase activity was defined as the amount of Lcc35 that oxidized 1 μmol substrate per min.

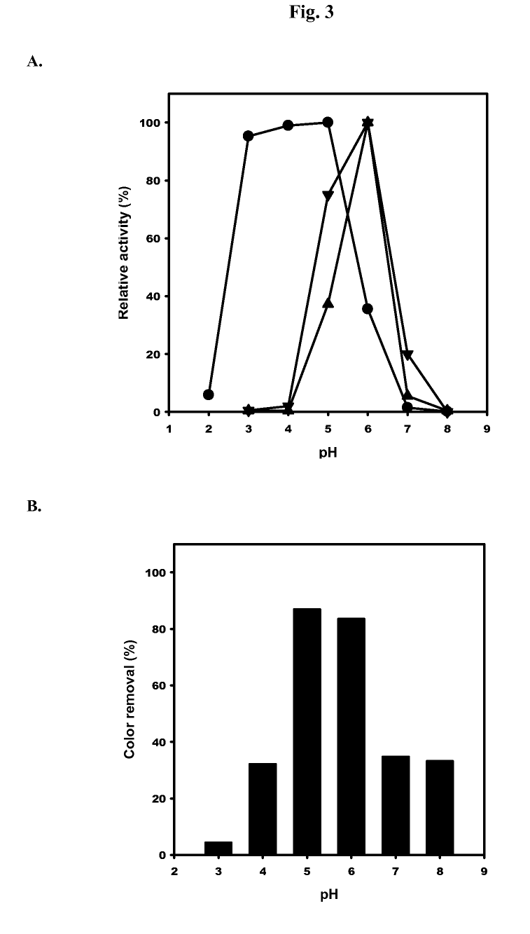

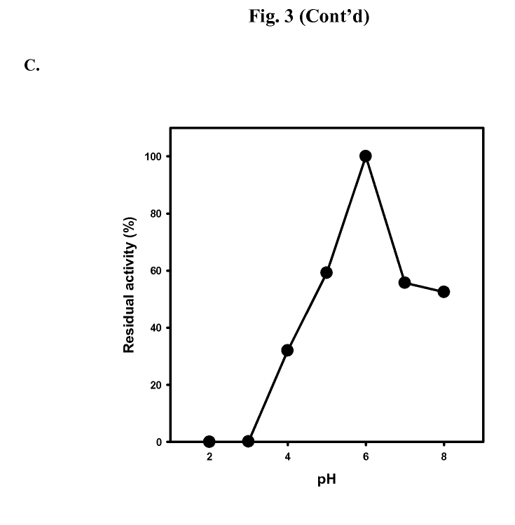

The results show that Lcc35 exhibited the highest laccase activity at pH 3-5 when ABTS was the substrate and at 5-6 when SGZ and RBBR were the substrates. See FIG. 3, panel A.

The optimal pH of Lcc35 was also examined using an artificial lignin as a substrate, as follows. Each of the reaction buffers listed above was mixed with 0.1% lignin (Sigma #471003) and the mixture was placed in wells of an agar micro-titter plate. 10 μl of the Lcc35-containing suspension was placed at the center of each well. The agar plate was incubated at 30° C. for 4 hr to allow degradation of the artificial lignin by Lcc35. The result indicates that, when using lignin as a substrate, the highest laccase was observed at pH 5-6. See FIG. 3, panel B.

The Lcc35 solution was incubated under various pH conditions at 30° C. 20 hours later, the laccase activity in the solution was determined, using ABST as the substrate. It has been found that Lcc35 was more stable under pH 6 than other pH conditions. See FIG. 3, panel C.

(iii) Optimal Reaction Temperature and Thermal Stability

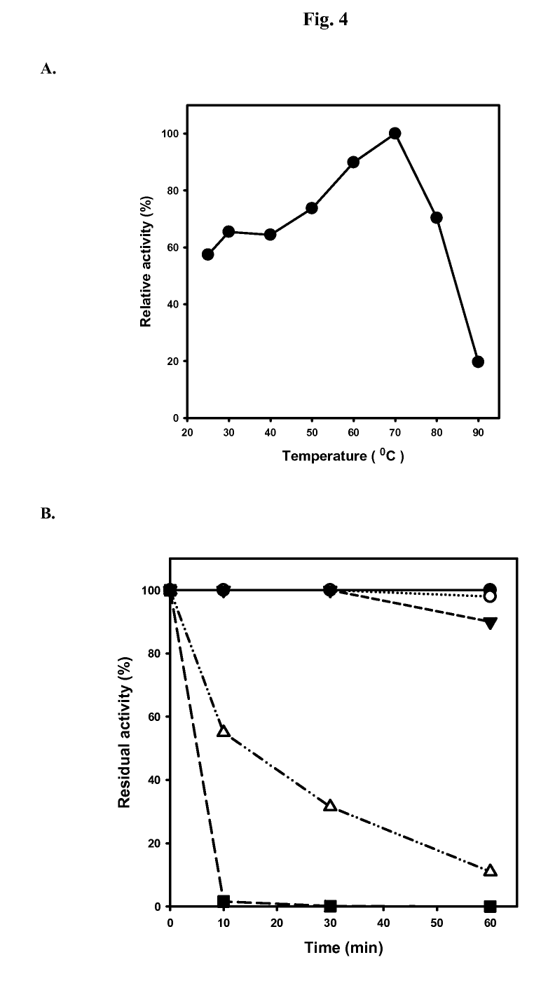

The laccase activity of Lcc35 was determined at various temperatures (i.e., ranging from 25-90° C.), using ABST as the substrate. The Lcc35-containing solution was incubated with ABST and a sodium acetate buffer (pH 5.0) for 1 minute and the enzymatic activity was examined afterwards. As shown in FIG. 4, panel A, the highest laccase activity was observed at 70° C.

To determine thermal stability of Lcc35, the enzyme solution was incubated under pH 6.0 at various temperatures (i.e., 30° C., 40° C., 50° C., 60° C., and 70° C.) for 10, 30, or 60 minutes. As shown in FIG. 4, panel B, Lcc35 was stable below 50° C.

### (iv) Kinetic Constants of Lcc35

Kinetic constants of Lcc35 for digesting ABTS and SGZ were determined based on the initial reaction rates of Lcc35 at various substrate concentrations. The enzymatic reactions were taken place at pH 5 (for ABST) or pH 6 (for SGH) and 70° C. The results were shown in Table 1 below:

TABLE 1
Kinetic properties of Lcc35 using two different substrates.

| | εₘₐₓ | Wavelength | Kₘ | K_{cat} | K_{cat}/Kₘ |
|---|---|---|---|---|---|
| Substrate | (M⁻¹ cm⁻¹) | (nm) | (μM) | (s⁻¹) | (μM⁻¹ s⁻¹) |
| ABTS | 36000 | 420 | 53 | 730 | 13.8 |
| SGZ | 65000 | 530 | 7 | 750 | 107.1 |

### (v) Inhibitor Effects

The inhibitory effects of NaN₃, ethylenediaminetetraacetic acid (EDTA), dithiothreitol (DTT), and L-Cysteine on Lcc35 were examined as follows. Lcc35 was pre-incubated in a solution (pH 5.0) containing one of the test compounds at 30° C. for 10 min. The laccase activity was then examined using ABTS as the substrate.

As shown in Table 2 below, NaN₃ substantially inhibited Lcc35 activity. By contrast, the other three test compounds displayed little inhibitory effect on Lcc35.

TABLE 2
Effect of inhibitors on Lcc35 activity.

| Compound | Concentration (mM) | Relative activity (%) |
|---|---|---|
| None | *** | 100.0 |
| NaN₃ | 0.1 | 50.0 |
| | 1.0 | 17.0 |
| EDTA | 10.0 | 100.0 |
| | 25.0 | 100.0 |
| DTT | 0.1 | 92.3 |
| | 1.0 | 91.8 |
| L-Cysteine | 0.1 | 97.2 |
| | 1.0 | 92.7 |

(vi) Substrate Specificity Comparison with Another Fungal Laccase

The laccase activity of Lcc35 was compared with Trametes versicolor laccase (provided by Fluka Co.), using either ABST or SGZ as the substrate according to the methods described above. The results show that the laccase-specific activity of Lcc35 was 2-3 fold higher than that of the T. versicolor laccase. See Table 3 below.

TABLE 3
Activity comparison between Lcc35 and T. versicolor laccase

| | | ABTS assay | SGZ assay |
|---|---|---|---|
| | Strain | (U mg⁻¹)^{a} | (U mg⁻¹)^{a} |
| Lcc35 Laccase | R. microporus BCRC | 3800 | 1700 |
| | 35318 | | |
| T. versicolor laccase | T. versicolor | 1300 | 500 |
| (Fluka co.) | | | |

^{a}The amount of laccases was analyzed on SDS-PAGE and calculated by ImageQuant TL 7.0 (GE Healthcare).

Cloning of lcc35 Gene from R. microporus

Lcc35 was subjected to N-terminal protein sequencing. The result indicates that the N-terminal fragment of Lcc35 has the amino acid sequence SVGPVADIP (SEQ ID NO:5). Degenerate primers listed in Table 4 below were designed for amplifying the gene that encodes Lcc35:

TABLE 4
Primer Sequences

| | | |
|---|---|---|
| Primer | Sequence | Purpose |

| | | |
|---|---|---|
| RT_polyT | ggttcttgccacagtcacgacttttttttttttttttt | poly (A) for RT |
| | (SEQ ID NO: 6) | |
| RT anchor | ggttcttgccacagtcacgac | 3′ RT anchor |
| | (SEQ ID NO: 7) | |
| Lcc35-2 | ggcccngtngcngayathcc | Degenerate primer for |
| | (SEQ ID NO: 8) | N-terminal sequence |
| Lcc35inverse_5′ | actcgtaccactgttctcgcaggtggaac | inverted PCR for |
| | (SEQ ID NO: 9) | 5′-end |
| Lcc35inverse_3′ | gaaaccatctggagagaggttagcgttg | inverted PCR for |
| | (SEQ ID NO: 10) | 3′-end |

Using the primers listed in Table 4 above, the genomic sequence and full-length cDNA sequence coding for Lcc35 were amplified from R. microporus BCRC 35318 via RT-PCR. As shown in FIG. 1, the full-length Lcc35 cDNA encodes a 515-amino-acid long polypeptide with a N-terminal signal peptide (1-21). The alignment of Lcc35 cDNA to its genomic DNA revealed that the lcc35 gene contains 13 exons and 12 introns. See FIG. 1.

### Example 2

### Preparation of Lcc35 in R. microporus BCRC 35318 in the Presence of Enhancers

R. microporus BCRC 35318 cells were cultured in the basal medium described in Example 1 above, which was supplemented with veratryl alcohol, 4-hydroxybenxoic acid, ferulic acid, or rice straw powder. Lcc35 were isolated from the culture supernatants following the method also described above and its activity was determined. The results are shown in Table 5 below:

TABLE 5
Effects of various inducers on Laccase production in R. microporus

| Inducer | Concentration | Yield (Unit ml⁻¹) |
|---|---|---|
| No inducer | — | 1.0 |
| Veratryl alcohol | 1 mM | 3.4 |
| 4-Hydroxybenzoic acid | 1 mM | 10.5 |
| Rice straws | 1 g 50 ml⁻¹ | 8.0 |
| Ferulic acid | 1 mM | 2.4 |

All of the inducers listed in Table 5 above enhanced Lcc35 production in R. microporus BCRC 35318. Among them, 4-hydroxybenzoic acid and rice straw increased Lcc35 production by 10.5-fold increase and 8.0 fold, respectively.

In the presence of 1 mM 4-hydroxybenzoic acid, R. microporus BCRC 35318 exhibited a higher laccase activity in a shorter cultivation period, as compared to known R. lignosus and P. pastoris strains that produce laccase. See Cambria et al., Protein Expr. Purif. 2000, 18, (2), 141-147; and Liu et al., Appl. Microbiol. Biotechnol. 2003, 63, (2), 174-181. Following the isolation process described in Example 1 above, purified Lcc35 was obtained with a recovery rate of about 55% and an enzymatic activity of 289.8 U/ml.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. An isolated polypeptide, comprising an amino acid sequence at least 85% identical to SEQ ID NO:1.

2. The polypeptide of claim 1, wherein the amino acid sequence is at least 90% identical to SEQ ID NO:1.

3. The polypeptide of claim 2, wherein the amino acid sequence is at least 95% identical to SEQ ID NO:1.

4. The polypeptide of claim 3, wherein the amino acid sequence is SEQ ID NO:1.

5. The polypeptide of claim 4, wherein the laccase has the amino acid sequence of SEQ ID NO:1.

6. The polypeptide of claim 4, wherein the laccase has the amino acid of SEQ ID NO:2.

7. An isolated nucleic acid, comprising a first nucleotide sequence encoding an amino acid sequence at least 85% identical to SEQ ID NO:1.

8. The isolated nucleic acid of claim 7, wherein the amino acid sequence is at least 90% identical to SEQ ID NO:1.

9. The isolated nucleic acid of claim 8, wherein the amino acid sequence is at least 95% identical to SEQ ID NO:1.

10. The isolated nucleic acid of claim 9, wherein the nucleotide sequence encodes SEQ ID NO:1.

11. The isolated nucleic acid of claim 10, wherein the nucleotide sequence is SEQ ID NO:3.

12. The isolated nucleic acid of claim 10, wherein the nucleic acid further contains a second nucleotide sequence linked to the 5′ end of the first nucleotide sequence, the second and first nucleotide sequences, taken together, encoding SEQ ID NO:2.

13. The isolated nucleic acid of claim 12, wherein the nucleic acid contains the nucleotide sequence of SEQ ID NO:4.

14. An expression vector, comprising the nucleic acid of claim 7.

15. A host cell, comprising the expression vector of claim 9.

16. A method of preparing the polypeptide of claim 1, comprising
providing a cell containing a nucleic acid for expressing the polypeptide of claim 1,
culturing the cell in a medium under conditions allowing expression of the polypeptide, and
collecting the cells, the medium, or both for isolation of the polypeptide.

17. The method of claim 16, wherein the polypeptide has the amino acid sequence of SEQ ID NO:1.

18. The method of claim 16, wherein the polypeptide has the amino acid sequence of SEQ ID NO:2.

19. The method of claim 16, further comprising, after the collecting step, determining laccase activity of the polypeptide.

20. The method of claim 16, wherein the cell is a R. microporus cell.

21. The method of claim 16, wherein the medium contains an inducer selected from the group consisting of 4-hydroxybenzoic acid, rice straw, veratryl alcohol, and ferulic acid.

22. The method of claim 21, wherein the inducer is 4-hydroxybenzoic acid or rice straw.
